(19) United States
(12) Patent Application Publication
(10) Pub. No.: US 2015/0150855 A1
(43) Pub. Date: Jun. 4, 2015

(54) METHOD OF IMPROVING LIVER FUNCTION

(71) Applicant: VERVA PHARMACEUTICALS LTD, Waum Ponds, Victoria (AU)

(72) Inventors: Ken Walder, Geelong (AU); Guy Krippner, Newton (AU); Geoff Nicholson, Geelong (AU)

(21) Appl. No.: 14/403,507

(22) PCT Filed: Mar. 15, 2013
(86) PCT No.: PCT/AU2013/000265

§ 371 Date: Nov. 24, 2014

Related U.S. Application Data
(60) Provisional application No. 61/651,335, filed on May 24, 2012.
(60) Provisional application No. 61/666,574, filed on Jun. 29, 2012.

(51) Int. Cl. A61K 31/433; A61K 31/155

(52) U.S. Cl. CPC A61K 31/433; A61K 31/155

(57) ABSTRACT

The present disclosure relates generally to the use of methazolamide in therapy. The disclosure further relates to treating liver dysfunction, or improving liver function, in a patient.

### FIELD

The present disclosure relates generally to the use of methazolamide in therapy. The disclosure further relates to treating liver dysfunction, or improving liver function, and/or lowering or decreasing ALT in a patient. The present disclosure further relates to the use of methazolamide and compositions and agents containing same, in treating liver dysfunction, or improving liver function, in a patient.

### BACKGROUND

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

Serum alanine transaminase, also known as alanine transaminase (ALT), is a transaminase enzyme found in high concentrations in the liver cytosol and at low concentrations elsewhere. ALT is released into the serum as a result of damage to the hepatic cells and as a result, elevated serum levels of ALT are typically (although not exclusively) considered to be a marker of hepatocellular injury or necrosis. Thus, ALT levels are usually elevated in a variety of hepatic diseases and disorders such as cirrhosis, hepatitis and damage due to drugs, toxins, and other medications. The normal reference range for ALT differs slightly between laboratories, but are typically reported in the ranges of about 0-40 U/L, and about 7-56 U/L. However, serum levels of ALT may fluctuate throughout the day, and are observed to increase in response to strenuous physical exercise or certain medications.

Hepatic steatosis is the deposition of triglycerides as lipid droplets in the cytoplasm of hepatocytes and reflects an imbalance between the uptake, synthesis and disposal of triglycerides by the liver. Steatosis may be defined as a hepatic triglyceride level exceeding the 95^{th} percentile for lean, healthy livers (i.e. >55 mg/g liver) or, more commonly, when intracellular lipids exceed 5% of the hepatic tissue. Evidence of steatosis is typically obtained either by imaging or histology.

The presence of hepatic steatosis, in the absence of other causes of secondary fat accumulation, such as significant alcohol consumption, use of steatogenic medication and/or hereditary factors, is diagnosed as non-alcoholic fatty liver disease (NAFLD).

NAFLD may be further categorized by histology into two subsets:
Non-alcoholic fatty liver (NAFL), where hepatic steatosis is present with no evidence of hepatocellular injury in the form of hepatocellular ballooning and cell death: and
Non-alcoholic steatohepatitis (NASH), where hepatic steatosis is present, along with inflammation and hepatocellular injury, with or without fibrosis (collagen deposition).

It is not clear whether steatosis always precedes NASH or whether NASH is a distinct disorder.

In many patients, simple steatosis (NAFL) is relatively benign. Patients with simple steatosis have very slow, if any, histological progression and patients are generally at a low risk for development of advanced disease.

NASH presents a significantly worse prognosis than NAFL and patients with NASH can exhibit histological progression to cirrhosis, liver failure and hepatocellular carcinoma. Between 10-29% of individuals with NASH develop cirrhosis within 10 years and 4-27% of individuals with NASH-induced cirrhosis develop hepatocellular carcinoma.

Patents with NASH have increased overall mortality compared with matched control populations (primarily through increased cardiovascular mortality); increased liver-related mortality; and increased risk of developing liver cancer. NASH with fibrosis has been shown to carry a worse prognosis than NASH without fibrosis. Fibrosis progression in NASH is associated with multiple metabolic factors, including diabetes mellitus, severe insulin resistance, elevated BMI, weight gain of greater than 5 kg and rising serum aminotransferase levels.

NAFLD is the most common cause of incidental elevation of liver enzymes in the Western world. The prevalence of NAFLD varies widely depending on the population studied; however, the median prevalence of NAFLD in the general population worldwide is 20% (range 6.3-33%). The estimated prevalence for NASH is lower, ranging from 3-5% of the general population. NAFLD prevalence is highest in non-white Hispanics, followed by Caucasians and non-Hispanic blacks. It is worth noting that the prevalence of NAFLD when estimated using aminotransferases (AST and ALT) alone, without imaging or histology, is only 7-11%, reflecting the fact that aminotransferase levels can be normal in individuals with NAFLD.

While the causes of liver disease are many, it is observed to be prevalent in patients having uncontrolled or higher than normal blood glucose levels, such as when pre-disposed to, or suffering from, a metabolic risk factor or metabolic disorder such as insulin resistance or diabetes. A broad spectrum of liver disease is seen in diabetic patients, including non-alcoholic fatty liver disease (NAFLD), cirrhosis, heptaocellular carcinoma, hepatitis and acute liver failure. In particular, NAFLD is highly associated with metabolic risk factors, including obesity (both excessive BMI and visceral obesity), and with metabolic disorders such as diabetes mellitus and dyslipidemia. NAFLD is observed in 60-76% of all diabetes patients and in 100% of diabetes patients who are also obese. NASH is present in at least 22% of diabetes patients. The presence of a metabolic disorder is a strong predictor of progression from NAFL to NASH. Patients with diabetic NASH have more severe inflammation and fibrosis on liver biopsy and tend to show faster progression to fibrosis than NASH patients without diabetes. Diabetes increases the risk of cirrhosis related complications from NASH and diabetic NASH patients have a 4-fold increase in the prevalence of hepatocellular carcinoma.

Diabetes is a metabolic disorder characterized by chronically elevated blood glucose levels (greater than about 126 mg/dL or 7.0 mmol/L). Blood glucose is derived from a combination of glucose absorbed from the diet and glucose produced by the liver and released into the blood stream (hepatic glucose production). Once entered into the blood stream, glucose requires the assistance of insulin to enter hepatic, muscle and adipose cells in order to be stored or utilised. Another′ major action of insulin is to suppress hepatic glucose production. In a healthy individual, glucose homeostasis is controlled primarily by insulin. As blood glucose levels rise, such as after eating, specialised β-cells within the pancreas release insulin which suppresses hepatic glucose production and promotes glucose uptake, intracellular metabolism and glycogen synthesis by the body's target tissues. Thus, in healthy individuals, blood glucose concentrations are strictly controlled, typically in the range of 80-110 mg/dl. However, where the pancreas produces an inadequate insulin response, or the target cells do not respond appropriately to the insulin produced, this results in a rapid accumulation of glucose in the blood stream (hyperglycemia).

High blood glucose levels over time may cause cardiovascular disease, retinal damage, renal failure, nerve damage, erectile dysfunction and gangrene (with the risk of amputation). Furthermore, in the absence of available glucose, cells turn to fats as an alternative energy source. Resulting ketones, a product of fat hydrolysis, can accumulate in the blood stream instigating hypotension and shock, coma and even death.

Chronically elevated blood glucose levels can arise from either inadequate insulin secretion (Type 1 diabetes) and/or an inadequate response or sensitivity of body tissues to insulin action (Type 2 diabetes). One of the primary diagnostic features of diabetes is the individual's loss of control over glucose homeostasis, so that post-prandial blood glucose levels remain elevated after meals and may remain high for extended periods of time. Diabetes may be characterised by persistent hyperglycemia, polyuria, polydipsia and/or hyperphagia, chronic microvascular complications such as retinopathy, nephropathy and neuropathy, and macrovascular complications, such as hyperlipidemia and hypertension which can lead to blindness, end-stage renal disease, limb amputation and myocardial infarction.

The three most common types of diabetes are type 1, type 2 and gestational.

Type 1 diabetes, known as insulin dependent diabetes mellitus (IDDM), or juvenile-onset diabetes, accounts for 10-15% of all diabetes cases. It is most commonly diagnosed in children and adolescents but can occur in young adults as well. It is characterised by p-cell destruction resulting in a loss of insulin secretory function. Most cases relate to autoimmune destruction of the β-cells. Treatment is via insulin injection and must be continued indefinitely.

Type 2 diabetes, known as non-insulin dependent diabetes mellitus (NIDDM) or late-onset diabetes, insulin levels are initially normal but the body's target cells lose their responsiveness to insulin. This is known as insulin resistance or insulin insensitivity. To compensate for this resistance, the pancreas secretes excess insulin. Over time, the pancreas becomes less able to produce enough insulin, resulting in chronic hyperglycemia. Initial symptoms of type 2 diabetes are typically milder than for type 1 and the condition may go undiagnosed for many years before more severe symptoms are observed. Lifestyle (smoking, pOor diet and inactivity) is considered to be the major determinant of type 2 diabetes incidence, although a genetic predisposition increases the risk of developing this disease.

Gestational diabetes occurs in about 2-5% of all pregnancies. It is temporary, but if untreated may cause foetal complications. Most sufferers make a complete recovery after the birth. However, a proportion of women who develop gestational diabetes go on to develop type 2 diabetes.

Other, less common, causes of diabetes include genetic defects in β-cells, genetically related insulin resistance, diseases of the pancreas, hormonal defects, malnutrition and chemical or drug influences.

Impaired glucose tolerance and impaired fasting glucose, are pre-type 2 diabetic states, closely related to type 2 diabetes, and occur when the blood glucose level is higher than normal, but not high enough to be classified as diabetes (about 100-125 mg/dL; 5.6-6.9 mmol/L). As with type 2 diabetes, the body produces insulin but in an insufficient amount or the target tissues are unresponsive to the insulin produced.

Impaired glucose tolerance, impaired fasting glucose and insulin resistance are components of Syndrome X, also known as Insulin Resistance Syndrome (IRS) or metabolic syndrome, which is a cluster of risk factors for heart disease that also includes: obesity, atherosclerosis, hypertriglyceridemia, low HDL cholesterol, hyperinsulinemia, hyperglycemia and hypertension.

The prevalence of type 2 diabetes has more than doubled over the last 2 decades and continues to grow at an alarming rate. The World Health Organization (WHO) estimates that 346 million people worldwide suffer from type 2 diabetes (approximately 4.9% of the world's population) with at least 50% of the diabetic population unaware of their condition (World Health Organization. Diabetes. Fact sheet No 312 August 2011, (www.who.int)). Another 7 million people are estimated to become diabetic each year. The increase in diabetes incidence worldwide is a particular concern in children: type 2 diabetes was diagnosed in 1-2% of children 30 years ago, but accounts for up to 80% of pediatric diabetes cases reported today. India currently has the highest number of diabetic persons, followed by China, the USA, Russia and Germany. Approximately 1.7 million Australians (7.5% of the population) have type 2 diabetes and 275 Australian adults become diabetic every day. Another 2 million Australians have pre-diabetes and are at risk of developing type 2 diabetes (Diabetes Australia—Vic (www.diabetesvic.org.au/health-professionals/diabetes-facts)). In the United States, an estimated 25.8 million people (8.3% of the population) have diabetes and a further 79 million are prediabetic (U.S. Department of Health and Human Services, Centers for Disease Control and Prevention (2011). National diabetes fact sheet: national estimates and general information on diabetes and prediabetes in the United States (www.cdc.gov/diabetes)). 1.9 Million new cases of adult diabetes are diagnosed in the US each year and at least one prediction has indicated that the current growth in diagnosed and undiagnosed diabetes means 50% of the US population could be diabetic or prediabetic by 2020 (UnitedHealth Group's Center for Health Reform & Modernization. The United States of Diabetes. Working paper 5. November, 2010). The economic costs of diabetes anti related conditions are dramatic. The estimated direct and indirect costs of diabetes to the Australian healthcare system are estimated to be at least AUD 3 billion. This is dwarfed by the US, where direct costs of diabetes were estimated to be USD 116 billion in 2007, with indirect costs accounting for an additional USD 58 billion. If the predicted increase in diabetes incidence in the US continues, the healthcare costs could reach USD 3.35 trillion (at least 10% of total health care spending).

Type 2 diabetes is ideally treated by lifestyle modification, particularly diet and exercise. Comprehensive clinical and epidemiological studies have demonstrated that weight loss of 5-11 kg can reduce diabetes risk by 50% and weight loss of ≧10 kg is associated with 30-40% decrease in diabetes-related deaths. Weight loss of 20-30 kg is curative of diabetes and hypertension in many patients (Labib M. (2003) The investigation and management of obesity. J Clin Pathol. 56: 17-25). Weight loss and exercise have also been shown to reduce liver enzyme levels and steatosis in obese patients (Bayard et al, American Family Physician, 73, 1961-1968, 2006).

Unfortunately, most patients cannot sustain such lifestyle modifications and pharmacological intervention is required for adequate glucose control. International treatment guidelines now include metformin with diet and exercise as the first-line therapy for type 2 diabetes (Inzucchi S E et al. (2012) Medical management of hyperglycemia in type 2 diabetes; a patient-centered approach. Position statement of the American Diabetes Association (ADA) and the European Association for the Study of Diabetes (EASD). Diabetes Care 35:1364-79; e-published ahead of print, 19 Apr. 2012). The multi-factorial nature of diabetes pathology means most patients will progress to combination therapy to maintain effective glucose control over their lifetime. If metformin and lifestyle modification are insufficient to establish glucose control, addition of a sulfonylurea, DPP4 inhibitor (such as sitagliptin), GLP-1 agonist (such as liraglutide) (second line) or three drug combinations (third line) are indicated. The thiazolidinedione (TZD) insulin sensitizers rosiglitazone and pioglitazone had previously been recommended as second-line therapy; however, significant safety concerns have severely limited their current use. Patients who cannot maintain glucose control with combination therapies will ultimately be required to use insulin. While insulin has previously been considered a last-line of diabetes therapy, physicians have become more willing to add basal insulin as a second-line therapy.

Current diabetes treatments are often limited by poor safety profiles. First-line therapy metformin causes gastrointestinal side-effects including dose-limiting diarrhea. Second-line therapy sulfonylureas (which increase insulin secretion), along with meglitinides, can cause dangerous hypoglycemia and accelerate pancreatic β-cell destruction. The sulfonylureas, meglitinides and metformin are all subject to tolerance and loss of efficacy over time. The TZD insulin sensitizers have been associated with severe edema, weight gain, bone fractures, cardiovascular side-effects (including increased risk of mortality from myocardial infarction), bladder cancer and increased risk of diabetic macular edema. Safety warnings have been issued for the DPP4 inhibitor sitagliptin regarding acute pancreatitis and the potentially fatal allergic reaction Stevens-Johnson Syndrome. The related molecule vildagliptin has been shown to elevate liver enzyme levels. Treatment with the GLP-1 agonist exenatide can cause nausea, pancreatitis and hypoglycemia. Development of antibodies to exenatide can also limit its utility in some patients. The GLP-1 agonist liraglutide has a high incidence of gastrointestinal side effects (including nausea and vomiting) and causes dose-dependent and treatment-duration-dependent thyroid C-cell tumors at clinically relevant exposures in rats and mice. Cost is also a significant issue with newer therapies. For example, sitagliptin is no more effective than metformin at lowering blood glucose levels but is 20-times more expensive (VanDeKoppel S et al. (2008) Managed care perspective on three new agents for type 2 diabetes. J Manag Care Pharm 14: 363-80).

The limitations identified for current non-insulin diabetes medicines means there is a pressing need to develop cost-effective new therapies with improved safety and efficacy profiles; high patient compliance; and potential to maintain/improve β-cell function and delay secondary treatment failure. There is a particularly a need for new, safe insulin sensitizers to replace the TZDs.

Pharmacologic therapy of diseases such as NAFLD, particularly patients suffering from or pre-disposed to a metabolic disease or risk factor, is a significant unmet medical need. In fact, there are no FDA approved treatments or guidelines for approving drugs for NAFLD.

There exists a need for new agents and treatments for patients suffering from liver disease, such as those patients who are also diabetic or pre-diabetic.

### SUMMARY

It has now been unexpectedly observed that administration of methazolamide can cause a decrease in serum ALT levels, thereby reflecting an improvement in liver function or the amelioration or treatment of liver disease. It has been shown for the first time that administration of methazolamide to diabetic patients, whether treated with another anti-diabetic agent or not, results in a reduction in serum ALT, a marker of liver disease or damage. It has also now surprisingly been shown that methazolamide is capable of reducing liver lipid levels. The use of methazolamide may therefore be a useful stand-alone or adjunctive (for example, for patients already established on anti-diabetic agents, such as metformin) treatment for liver dysfunction and disease and may advantageously further treat a diabetic or pre-diabetic condition or disorder in a patient by ameliorating insulin resistance, and/or maintaining normal or lowering elevated blood glucose levels.

Thus, in an embodiment the present disclosure relates to a method of decreasing serum ALT levels, in a patient in need thereof comprising administering an effective amount of methazolamide to said patient.

In an embodiment the present disclosure also relates to a method of treating or preventing liver dysfunction in a patient in need thereof, comprising administering an effective amount of methazolamide to said patient.

In further embodiments the present disclosure also relates to a method for reducing liver lipid content in patient in need thereof, comprising administering an effective amount of methazolamide to said patient.

In further embodiments, the disclosure relates to the treatment of liver disease, such as NAIL or treatment or prevention of NASH or NASH with fibrosis. Thus in some embodiments the disclosure also relates to a method for treating or preventing liver disease, such as NAFL or NASH, in a patient in need thereof, comprising administering an effective amount of methazolamide to said patient.

In further embodiments the present disclosure also relates to the use of methazolamide in the manufacture of a medicament. In some embodiments, the medicament is for decreasing serum ALT levels and/or treating or preventing liver dysfunction, and/or reducing elevated liver lipid levels and/or treating or preventing liver disease in a patient.

The disclosure also relates to methazolamide for use in therapy. In some embodiments, the therapy is for decreasing serum ALT levels and/or treating or preventing liver dysfunction, and/or reducing elevated liver lipid levels and/or treating or preventing liver disease in a patient.

In some embodiments:
(a) the patient has elevated ALT levels, such as greater than about 50 U/L, for example, ≧80 U/L or ≧100 U/L or ≧200 U/L; and/or
(b) the patient is suffering from liver dysfunction, which may be symptomatic or asymptomatic; and/or
(c) the patient is susceptible to or suffering from a pre-diabetic or diabetic condition

In some embodiment thereof, the patient for treatment has an initial haemoglobin A_{1c} (HbA_{1c}) level of ≧6.5%. In some embodiments, the therapy of the disclosure lowers or controls the haemoglobin A_{1c} (HbA_{1c}) level to 6.5% or below.

In further embodiments, the patient suffers form one or more of (a), (b) or (c) as above, for example, in some embodiments the patient may present with one or both of (a) and (b), but not (c). In other embodiments, the patient may present with (a) and/or (b), and may further be susceptible to or suffer from a pre-diabetic or diabetic condition (c). In other embodiments, the patient does not present with (a) or (b) but is susceptible to or suffering from a pre-diabetic or diabetic condition (c).

Pre-diabetic and diabetic conditions referred to herein include impaired glucose tolerance, impaired fasting glucose and insulin resistance, Syndrome X, also known as Insulin Resistance Syndrome (IRS) or metabolic syndrome, type 2 diabetes and risk factors such as obesity, atherosclerosis, hypertriglyceridemia, low HDL cholesterol, hyperinsulinemia, hyperglycemia and hypertension. In some embodiments, the treatment with methazolamide is concurrent with treatment with an anti-diabetic agent, such as metformin.

In further embodiments, the patient has been previously commenced on and is undergoing treatment with an anti-diabetic agent.

The present disclosure further relates to compositions for decreasing serum ALT levels and/or treating or preventing liver dysfunction, and/or reducing elevated liver lipid levels and/or treating or preventing liver disease in a patient, comprising methazolamide together with one or more pharmaceutically acceptable additives.

The present disclosure also relates to a combination for decreasing serum ALT levels and/or treating or preventing liver dysfunction, and/or reducing elevated liver lipid levels and/or treating or preventing liver disease in a patient suffering from a diabetic or pre-diabetic condition, said combination comprising methazolamide and an anti-diabetic agent. The combination may be presented as separate formulations to be administered separately, simultaneously or sequentially, or formulated as a single unitary dosage.

Further embodiments relate to the use of methazolamide in treating liver diseases such as NAFLD, for example, NAFL or NASH, with or without fibrosis.

In some embodiments, the methazolamide is administered to the patient in an amount less than 100 mg per day, such as about 90, 85, 80, 75, 70, 65, 60, 55 or 50 mg per day, either as a single dose or a divided dose

To some embodiments, the anti-diabetic agent is an insulin sensitiser, such as metformin, or a pharmaceutically acceptable salt thereof, for example metformin hydrochloride.

### DESCRIPTION OF THE FIGURES

FIG. 1 graphically depicts the effect of methazolamide treatment in reducing the serum alanine aminotransferase (ALT) levels in diabetes patients who are not receiving any other diabetes medicines or have been stable on metformin for at least 3 months prior to methazolamide treatment.

FIGS. 2(A)-(D) depict liver lipid levels in vehicle treated db/db mice.

FIGS. 3(A)-(D) depict liver lipid levels in methazolamide treated db/db mice.

### DESCRIPTION

Throughout this, specification and the claims which follow, unless the context requires otherwise, the word “comprise” and variations such as “comprises” and “comprising” will be understood to imply the inclusion of a stated integer or step or group of integers but not the exclusion of any other integer or step or group of integers.

Throughout this specification and the claims which follow, unless the context requires otherwise, the phrase “consisting essentially of”, and variations such as “consists essentially of” will be understood to indicate that the recited element(s) is/are essential i.e. necessary elements of the invention. The phrase allows for the presence of other non-recited elements which do not materially affect the characteristics of the invention but excludes additional unspecified elements which would affect the basic and novel characteristics of the method defined.

The singular forms “a”, “an” and “the” include plural aspects unless the context clearly dictates otherwise.

The term “invention” includes all aspects, embodiments and examples as described herein.

A patient as contemplated herein may have normal or elevated ALT levels. In some embodiments, the patient presents with elevated ALT levels, being levels at least above the upper limit of normal (ULN), i.e. approximately ≧50 U/L. Examples of elevated ALT levels include those in the range of about 50-100 U/L (e.g. about 70 U/L or greater), or about 100-200 U/L or about 250-500 U/L. In severe or advanced liver disease ALT levels may exceed 1000 or 2000 U/L, i.e. elevated ALT levels can be about 1.5, 2-3 or 4-5 or 10-20, or 50-100 times ULN. However, even patients with normal ALT levels may have underlying liver disease or dysfunction. In accordance with the present disclosure a patient may or may not have elevated ALT levels.

Liver dysfunction, as used herein is intended to encompass the presence of hepatic (liver) disease, wherein hepatic tissue may be damaged, and/or where normal liver function is compromised, and includes the following conditions: NAFLD (such as steatosis (elevated liver lipid levels NASH, and NASH with fibrosis), cirrhosis, hepatitis (e.g. B or C), steatohepatitis, liver damage by alcohol, toxins or medication, inflammation, necrosis and fibrosis of the liver, acute liver failure and hepatocellular carcinoma. In some embodiments, the disclosure herein thus relates to treating or preventing liver dysfunction. A patient suffering from liver dysfunction may be symptomatic (present symptoms, such as elevated ALT levels) of liver dysfunction, or on the other hand, be asymptomatic. The presence of liver disease can be established by methods known in the art therefor, such as testing for elevated levels liver enzymes, (e.g. ALT and/or aspartate transaminase (AST), and/or liver biopsy, and/or imaging techniques, such as ultrasound, nuclear magnetic resonance and computer tomography). Thus, in some embodiments, the disclosure provides for the treatment or prevention of liver disease, such as described herein, in a patient, for example the treatment of NAFLD.

Treatment of liver dysfunction or disease is intended to include the amelioration, halting or slowing of progression, reversal or otherwise improvement in liver function or the pathology or any other symptom(s) associated with the underlying condition.

As used herein, elevated liver lipid levels includes levels of about or greater than 55 mg/g liver, or greater than about 5% of hepatic tissue.

Methazolamide is approved for use in the treatment of ocular conditions where lowering intraocular pressure is likely to be of therapeutic benefit, such as chronic open-angle glaucoma, secondary glaucoma, and preoperatively in acute angle-closure glaucoma where lowering the intraocular pressure is desired before surgery. Methazolamide exerts its effect on ocular conditions through inhibition of the enzyme carbonic anhydrase; however, this does not appear to be the mechanism responsible for its activity as an insulin sensitizer in diabetes. The therapeutically effective (carbonic anhydrase inhibitory) intraocular pressure-reducing dose of methazolamide is in the range of from 50 mg to 100-150 mg, 2 or 3 times daily, i.e. from 100-450 mg per day. Some metabolic acidosis and electrolyte imbalance may occur with the use of carbonic anhydrase inhibitory effective amounts, but excessive acidosis which, can lead to a symptom complex of malaise, fatigue, weight loss, depression and anorexia, can occur at dosage amounts at the lower end of the standard dosage range (Epstein and Grant, Arch. Opthamol., 95, 1380, 1977). Although commonly described as a diuretic, it has only a weak and transitory diuretic activity, and product labelling specifically states that it should not be used as a diuretic.

In accordance with the disclosure, methazolamide is administered in an amount effective to achieve the desired level of therapeutic treatment or prevention, for example, in an amount effective to lower ALT levels and/or treat or prevent liver dysfunction, according to a desired dosing regime as determined by the attending physician. In some embodiments, the amount administered is also sufficient to reduce elevated blood glucose levels or maintain normal or desired blood glucose levels, either alone or in conjunction with one or more anti-diabetic agents, for example, in a synergistic or additive manner with the one or more anti-diabetic agents. In some embodiments, the therapeutic effects of methazolamide as disclosed herein can be achieved by dosage amounts such that they avoid or minimise clinically meaningful carbonic anhydrase inhibition, such as required for therapeutic treatment of ocular conditions, and also the dosages used avoid or minimise clinically meaningful acidosis which may be associated with standard carbonic anhydrase inhibitory effective dosage regimes. Thus, in some embodiments, methazolamide is advantageously administered to the patient at a dosage rate of less than 100 mg per day. In further embodiments, the methazolamide is administered at a dosage rate of about, 90, 85, 80 or 75 mg or less per day, or about 70, 65, 60, 55 or 50 mg or less per day. In still further embodiments, the methazolamide is administered at a dosage rate of about 40 mg or less per day. In yet further embodiments the methazolamide is administered at a dosage rate of about 30 mg or less per day. In yet further embodiments the methazolamide is administered at a dosage rate of about 25 mg or less per day. In still further embodiments the methazolamide is administered at a dosage rate of about 20 mg or less per day, such as about 15, 10 or 5 mg per day. Administration of any of these dosage amounts may be once a day, as a single dose, or a divided dose, such as twice or thrice a day or according to any other dosing regime as determined by the attending physician. Suitable unit dosages of methazolamide may contain about 1.0, 2.5, 5.0, 10, 20, 25, 30, 40, 50, 60, 75, 80 or 90 mg of methazolamide.

In some embodiments, the patients contemplated herein also suffer from a diabetic or pre-diabetic condition, which includes any disease or condition, or symptom or causative factor thereof in which insulin resistance or impaired glucose uptake by a cell or tissue can be attributed, or play a role or is manifested, and for which treatment with an anti-diabetic agent (also referred to herein as an anti-hyperglycemic agent) is prescribed for treatment. Non-limiting examples thereof include NIDDM (type 2 diabetes), gestational diabetes, impaired glucose tolerance, impaired fasting glucose, Syndrome X, hyperglycemia, atherosclerosis, hypertriglyceridemia, dyslipidemia, hyperinsulinemia, nephropathy, neuropathy, ischemia, and stroke.

Thus, in some embodiments, patients contemplated by the disclosure have been diagnosed as suffering from or susceptible to conditions as contemplated above and may be established on a treatment regime for that condition, such as with an anti-diabetic agent (e.g metformin). In some embodiments, said patient has commenced treatment at least 1 or 2 weeks prior to commencement of methazolamide treatment. In further embodiments the patient has commenced treatment at least 4 weeks (or 1 month) prior to commencement of methazolamide treatment. In still further embodiments the patient has commenced treatment at least 6, 8, 10 or 12 weeks (for example at least about 2 or about 3 months) prior to commencement of methazolamide treatment. In some embodiments it is advantageous for the patient to have been stabilised on the anti-diabetic agent prior to commencement of methazolamide treatment, that is to say, a dosing regime has been determined and commenced such that a stable desired blood glucose level, as determined by the attending physician has been achieved. Blood glucose levels can be measured by any suitable means typically used in the art, e.g. fasting blood glucose, HbA_{1c} levels etc. Exemplary stabilised levels include HbA_{1c} levels of 6.5% or less or fasting state blood glucose levels less than about 6.1 mmol/L (110 mg/dL).

In some embodiments, the methazolamide is administered in the absence of an adjunctive anti-diabetic agent, whether the patient is suffering from a diabetic or pre-diabetic condition or not. Thus, in some embodiments, the methods, medicaments, combinations and compositions herein consist essentially of methazolamide for administration to said patient.

Agents for the treatment of conditions associated with the diabetic and pre-diabetic state, such as cardiovascular disease (e.g. antihypertensive agents, anti-dyslipidemic agents), may also be administered in conjunction (simultaneously or separately) with methazolamide (and optionally an anti-diabetic agent). Any such associated symptoms or conditions may be treated with an appropriate agent, e.g. anti-hypertensive such as diuretics, ACE inhibitors or β-blockers as determined by the attending physician. In some embodiments, the disclosure herein may advantageously obviate the need for or reduce the dosage amount of such agents. It will be understood therefore that a patient may not necessarily suffer from or develop all symptoms or conditions associated with a diabetic or pre-diabetic disease or condition or, the condition may not be severe enough to warrant additional therapeutic treatment particularly if the disease or condition is detected and treated at an early stage.

In some embodiments the methazolamide may be administered in combination, either separately, simultaneously or sequentially with one or more other agents for decreasing serum ALT levels and/or treating or preventing liver dysfunction, and/or reducing elevated liver lipid levels and/or treating or preventing liver disease in a patient, such as vitamin E and/or other antioxidants. In some embodiments, there is provided a composition or combination of methazolamide and an antioxidant, for example vitamin E.

In embodiments where methazolamide is administered in conjunction with a treatment regime using another anti-diabetic therapeutic agent, the methazolamide may be co-administered simultaneously with, or sequentially to (before or after), the anti-diabetic therapeutic agent, and in the case of simultaneous administration, each agent may be formulated separately, or alternatively, both are formulated together into an intimate composition. Suitable anti-diabetic agents may include insulin sensitisers, insulin secretagogues glucose resorption/uptake inhibitors and the classes and compounds identified in US2005/0037981, particularly Table 2, the contents of which are incorporated herein in their entirety. Some examples of agents for use include biguanides, sulfonylureas, meglitinides, insulin and insulin analogues, and thiazolidinediones. Further non-limiting examples include thiazolidinediones (including rosiglitazone and pioglitazone), metformin and pharmaceutically acceptable salts thereof; such as hydrochloride, insulin, sulphonylureas (including glimepiride, glyburide, glipizide, chlorpropamide, tolazamide and tolbutamide), meglitimides (including repaglinide and nateglinide), α-glucosidase inhibitors (including a carbose and miglitol), GLP analogues such as exenatide and DPPIV inhibitors such as sitagliptin.

In some embodiments, the anti-diabetic agent metformin or a pharmaceutically acceptable salt thereof.

In some embodiments, by co-administering methazolamide once the patient is established on a treatment with an anti-diabetic agent, such as metformin, it may be possible to subsequently reduce the dosage of the anti-diabetic agent compared to the initial monotherapy. This may advantageously avoid, ameliorate, or otherwise reduce the severity, risk or occurrence of undesirable side effects and disadvantages associated with dosage amounts and regimes employed for the monotherapy. Thus, in some embodiments, the dosage regime of the anti-diabetic agent commenced prior to methzolamide treatment may be adjusted once methazolamide treatment is commenced or has been undertaken for a period of time.

As used herein, the terms “regulate” or “modulate” and variations such as regulating/modulating and regulation/modulation, when used in reference to glucose homeostasis, refer to the adjustment or control of said glucose levels, in particular embodiments, the adjustment to or maintenance of normal blood glucose levels. Thus, “regulating/modulating glucose homeostasis” includes the adjustment or control of blood glucose levels to lower hyperglycaemic, or advantageously achieve or maintain normal fasting state, blood glucose levels. Normal fasting state blood glucose levels are typically less than 6.1 mmol/L (110 mgd/L). Hyperglycemic levels (also referred to herein as elevated blood glucose levels) refer to fasting blood glucose levels greater than or equal to 6.1 mmol/L (110 mgd/L).

Impaired fasting glycemia (IFG) is characterised by a fasting plasma glucose concentration greater than or equal to 6.1 mmol/L (110 mgd/L) but less than 7.0 (126 mgd/L) and a 2-h plasma glucose concentration during the oral glucose tolerance test (OGTT) (if measured) less than 7.8 mmol/L (140 mgd/L). Impaired glucose tolerance (IGT) is characterised by a fasting plasma glucose concentration of less than 7.0 mmol/L (126 mgd/L) and a 2-h plasma glucose concentration during the OGTT of greater than or equal to 7.8 mmol/L (140 mgd/L) but less than 11.1 mmol/L (200 mgd/L). Diabetes is characterised by a fasting plasma glucose concentration of greater than or equal to 7.0 mmol/L (126 mgd/L); or a 2-h plasma glucose concentration during the OGTT of greater than 11.1 mmol/L (200 mgd/L); or a haemoglobin A_{1c} (HbA1c) level ≧6.5%. In some embodiments, the patient has a haemoglobin A_{1c} (HbA1c) level ≧7.0%. Treatment in accordance with the disclosure may also reduce blood glucose levels, especially in a diabetic or pre-diabetic patient. Thus, in some embodiments, treatment in accordance with the disclosure results in a haemoglobin A_{1c} (HbA1c) level less than about 6.5%, e.g. about 6.4-6.0% or less.

Patients contemplated herein include mammalian subjects: humans, primates, livestock animals (including cows, horses, sheep, pigs and goats), companion animals (including dogs, cats, rabbits, guinea pigs), and captive wild animals. Laboratory animals such as rabbits, mice, rats, guinea pigs and hamsters are also contemplated as they may provide a convenient test system. Human patients are particularly contemplated.

As described above, combinations according to the invention using another anti-diabetic agent, such as metformin, or a pharmaceutically acceptable salt thereof, may advantageously allow for reduced dosage amounts of said agent compared to known therapies for that agent, particularly monotherapy. In some embodiments, the dosage amounts of the combinations are such that they may provide an additive or synergistic effect. Suitable dosage amounts and dosing regimens can be determined by the attending physician and may depend on the particular condition being treated, the severity of the condition as well as the general age, health and weight of the subject.

In some embodiments of the disclosure, where the anti-diabetic agent is metformin, the daily dosage amount of metformin (or pharmaceutically acceptable salt, such as the hydrochloride) administered in the combination is equal to or less than about 90% of that which would be required for metformin monotherapy. In further embodiments, the dosage is equal to or less than about 80%, 70%, 60% or 50% of that which would be required for metformin monotherapy. Exemplary daily dosage amounts of metformin for an adult may be in the range of from about 100 mg to about 1500 or 2000 mg of active per day, such as 1.5 about 250 mg, 500 mg, 750 mg, 850 mg, 1000 mg, 1100 or 1250 mg. Exemplary daily dosage amounts for pediatric patients (10-16 years) may be in the range from about 50, to about 1000 mg or 1500 mg per day, such as about 100 mg, 250 mg, 500 mg, 750 mg, 850 mg, 1100 mg or 1250 mg per day. The active ingredient may be administered in a single dose or a series of doses. Suitable dosages forms may contain about 50, 75, 100, 150, 200, 250, 500 750, 850 or 1000 mg of metformin active.

While methazolamide and, optionally the anti-diabetic agent may be administered in the absence of any other agents or additives, it is preferable to present each, or an intimate composition thereof, as a composition with one or more pharmaceutically acceptable additives.

The formulation of such compositions is well known to those skilled in the art, see for example, Remington's Pharmaceutical Sciences, 21^{st} Edition. The composition may contain any suitable additives such as carriers, diluents or excipients. These include all conventional solvents, dispersion media, fillers, solid carriers, coatings, antifungal and antibacterial agents, dermal penetration agents, surfactants, isotonic and absorption agents and the like. It will be understood that the compositions of the invention may also include other supplementary physiologically active agents.

The carrier must be pharmaceutically acceptable in the sense of being compatible with the other ingredients of the composition and not injurious to the subject. Compositions include those suitable for oral, rectal, inhalable, nasal, topical (including dermal, buccal and sublingual), vaginal or parental (including subcutaneous, intramuscular, intravenous and intradermal) administration. The compositions may conveniently be presented in unit dosage form and may be prepared by any methods well known in the art of pharmacy.

Compositions of the present disclosure suitable for oral administration may be presented as discrete units such as capsules, sachets or tablets each containing a predetermined amount of the active ingredient; as a powder or granules; as a solution or a suspension in an aqueous or non-aqueous liquid; or as an oil-in-water liquid emulsion or a water-in-oil liquid emulsion.

A tablet may be made by compression or moulding, optionally with one or more accessory ingredients. Compressed tablets may be prepared by compressing in a suitable machine the active ingredient in a free-flowing form such as a powder or granules, optionally mixed with a binder (e.g. inert diluent), preservative disintegrant (e.g. sodium starch glycolate, cross-linked polyvinyl pyrrolidone, cross-linked sodium carboxymethyl cellulose) surface-active or dispersing agent. Moulded tablets may be made by moulding in a suitable machine a mixture of the powdered compound moistened with an inert liquid diluent. The tablets may optionally be coated or scored and may be formulated so as to provide slow or controlled release of the active ingredient therein using, appropriate coatings, for example, hydroxypropylmethyl cellulose in varying proportions to provide the desired release profile. Tablets may optionally be provided with an enteric coating, to provide release in parts of the gut other than the stomach.

Compositions suitable for parenteral administration include aqueous and non-aqueous isotonic sterile injection solutions which may contain anti-oxidants, buffers, bactericides and solutes which render the composition isotonic with the blood of the intended recipient; and aqueous and non-aqueous sterile suspensions which may include suspending agents and thickening agents. The compositions may be presented in unit-dose or multi-dose sealed containers, for example, ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules and tablets of the kind previously described.

It should be understood that in addition to the active ingredients particularly mentioned above, the compositions of this disclosure may include other agents conventional in the art having regard to the type of composition in question, for example, those suitable for oral administration may include such further agents as binders, sweeteners, thickeners, flavouring agents disintegrating agents, coating agents, preservatives, lubricants and/or time delay agents. Suitable sweeteners include sucrose, lactose, glucose, aspartame or saccharine. Suitable disintegrating agents include corn starch, methylcellulose, polyvinylpyrrolidone, xanthan gum, bentonite, alginic acid or agar. Suitable flavouring agents include peppermint oil, oil of wintergreen, cherry, orange or raspberry flavouring. Suitable coating agents include polymers or copolymers of acrylic acid and/or methacrylic acid and/or their esters, waxes, fatty alcohols, zein, shellac or gluten. Suitable preservatives include sodium benzoate, vitamin E, alpha-tocopherol, ascorbic acid, methyl paraben, propyl paraben or sodium bisulphite. Suitable lubricants include magnesium stearate, stearic acid, sodium oleate, sodium chloride or talc. Suitable time delay agents include glyceryl monostearate or glyceryl distearate.

Compounds for administration in accordance with the disclosure may optionally be presented as a pharmaceutically acceptable salt or prodrug as appropriate.

The term “prodrug” is used in its broadest sense and encompasses those derivatives that are converted in vivo, either enzymatically or hydrolytically, to the compounds of the invention. Such derivatives would readily occur to those skilled in the art, and include, for example, compounds where a free thiol or hydroxy group is converted into an ester, such as an acetate, or thioester or where a free amino group is converted into an amide. Procedures for acylating the compounds of the invention, for example to prepare ester and amide prodrugs, are well known in the art and may include treatment of the compound with an appropriate carboxylic acid, anhydride or chloride in the presence of a suitable catalyst or base. Esters of carboxylic acid (carboxy) groups are also contemplated. Suitable esters include C₁₋₆alkyl esters; C₁₋₆alkoxymethyl esters, for example methoxymethyl or ethoxymethyl; C₁₋₆alkanoyloxymethyl esters, for example, pivaloyloxymethyl; phthalidyl esters; C₃₋₈cycloalkoxycarbonyl C₁₋₆alkyl esters, for example, 1-cyclohexylcarbonyloxyethyl; 1,3-dioxolen-2-onylmethyl esters, for example, 5-methyl-1,3-dioxolen-2-onylmethyl; and C₁₋₆alkoxycarbonyloxyethyl esters, for example, 1-methoxycarbonyloxyethyl. Prodrugs of amino functional groups include amides (see, for example, Adv. BioSci., 1979, 20, 369, Kyncl, J. et al), enamines (see, for example, J. Pharm. Sci., 1971, 60, 1810, Caldwell, H. et al), Schiff bases (see, for example, U.S. Pat. No. 2,923,661 and Antimicrob. Agents Chemother., 1981, 19, 1004, Smyth, R. et al), oxazolidines (see, for example, J. Pharm. Sci, 1983, 72, 1294, Johansen, M. et al), Mannich bases (see, for example, J Pharm. Sci. 1980, 69, 44, Bundgaard, H. et al and J. Am. Chem. Soc., 1959, 81, 1198, Gottstein, W. et al), hydroxymethyl derivatives (see, for example, J Pharm. Sci, 1981, 70, 855, Bansal, P. et al) and N-(acyloxy)alkyl derivatives and carbamates (see, for example, J. Med. Chem., 1980, 23, 469, Bodor, N. et al, J. Med. Chem., 1984, 27, 1037, Firestone, R. et al, J. Med. Chem., 1967, 10, 960, Kreiger, M. et al, U.S. Pat. No. 5,684,018 and J. Med. Chem., 1988, 31, 318-322, Alexander, J. et al). Other conventional procedures for the selection and preparation of suitable prodrugs are known in the art and are described, for example, in WO 00/23419; Design of Prodrugs, H. Bundgaard, Ed., Elsevier Science Publishers, 1985; Methods in Enzymology, 42: 309-396, K. Widder, Ed, Academic Press, 1985; A Textbook of Drug Design and Development, Krogsgaard-Larsen and H. Bundgaard, Eds, Chapter 5, p 113-191 (1991); Advanced Drug Delivery Reviews, 8; 1-38 (1992); Journal of Pharmaceutical Sciences, 77; 285 (1988), H. Bundgaard, et al; Chem Pharm Bull, 32692 (1984), N. Kakeya et al and The Organic Chemistry of Drug Desig and Drug Action, Chapter 8, pp 352-401, Academic press, Inc., 1992.

Suitable pharmaceutically acceptable salts include, but are not limited to salts of pharmaceutically acceptable inorganic acids such as hydrochloric, sulphuric, phosphoric nitric, carbonic, boric, sulfamic, and hydrobromic acids, or salts of pharmaceutically acceptable organic acids such as acetic, propionic, butyric, tartaric, maleic, hydroxymateic, fumaric, maleic, citric, lactic, mucic, gluconic, benzoic, succinic, oxalic, phenylacetic, methanesulphonic, toluenesulphonic, benezenesulphonic, salicyclic sulphanilic, aspartic, glutamic, edetic, stearic, palmitic, oleic, lauric, pantothenic, tannic, ascorbic, fendizoic, 4-4′-methylenebis-3-hydrOxy-2-naphthoic acid, 0-(p-hydroxybenzoyl)benzoic, 4′-4″-dihydroxytriphenylmethane-2-carboxylic acid and valeric acids. Base salts include, but are not limited to, those formed with pharmaceutically acceptable cations, such as sodium, potassium, lithium, calcium, magnesium, ammonium and alkylammonium. Basic nitrogen-containing groups may be quaternised with such agents as lower alkyl halide, such as methyl, ethyl, propyl, and butyl chlorides, bromides and iodides; dialkyl sulfates like dimethyl and diethyl sulfate; and others.

The compounds of the invention may also be presented for use in veterinary compositions. These may be prepared by any suitable means known in the art. Examples of such compositions include those adapted for:

oral administration, e.g. tablets, boluses, powders, granules, pellets for admixture with feedstuffs, pastes for application to the tongue, drenches including aqueous and non-aqueous solutions or suspensions;

parenteral administration, e.g. subcutaneous, intramuscular or intravenous injection as a sterile solution or suspension.

The invention will now be described with reference to the following examples which are provided for the purpose of illustrating some embodiments of the invention and are not to be construed as limiting the generality hereinbefore described.

### EXAMPLES

### Example 1

### Effects of Methazolamide on ALT Levels in Type 2 Diabetic Patients

The safety and efficacy of methazolamide (40 mg administered twice daily) as a potential treatment for type 2 diabetes were evaluated in a 24 week, randomised, placebo-controlled double-blind clinical trial. The primary efficacy endpoint for the clinical trial was a reduction in HbA_{1c} (ΔHbA_{1c}) from baseline with methazolamide, relative to placebo, after 24 weeks of treatment. The primary safety measurement was the effect of methazolamide, compared to placebo, on venous blood gas parameters; a measure of acidosis.

The clinical trial initially enrolled type 2 diabetes patients who were not treated with any anti-diabetic agent prior to entry into the trial. The trial was expanded to include participants who had been treated with metformin for at least 3 months and were on a stable metformin dose for at least 8 weeks prior to entering the trial (MET). The metformin dose was not altered throughout the trial. Participant baseline demographic data are provided in Table 1-1,

Participants randomized into the clinical trial were administered either daily doses of methazolamide (40 mg b.i.d.) or placebo for 24 weeks. Methazolamide was taken as 1×30 mg capsule and 1×10 mg capsule per dose at breakfast and dinner. Placebo (microcrystalline cellulose) was administered in identical presentation. After an initial randomization visit to the clinic (Day 0), participants returned to the clinic at weeks 1, 2, 4, 8, 12, 18 and 24 for physical examinations, laboratory analyses, body composition measures, evaluation of glycemic parameters (fasting blood glucose, fasting insulin, HbA_{1c}) and measurement of venous blood gas analysis.

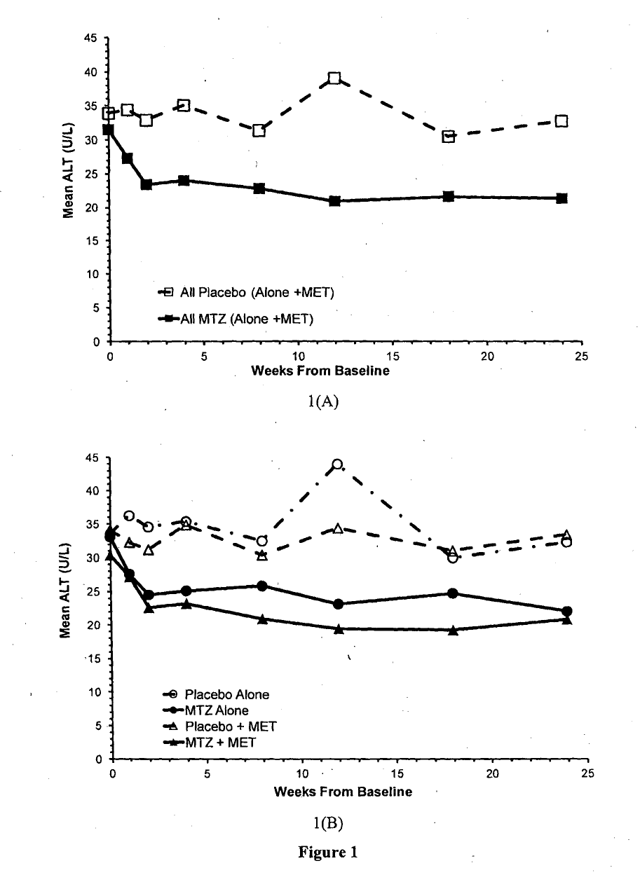

The effects of methazolamide on ALT are presented in Table 1-2. Mean ALT levels over time are depicted in FIGS. 1(A) and 1(B).

Surprisingly, patients treated with methazolamide showed a reduction in blood ALT levels that was evident after 1 week of methazolamide treatment. The reduced ALT level reached a plateau after 2 weeks of treatment that was maintained for the remainder of the 24 week treatment period. The methazolamide effect on ALT, and potential methazolamide action to treat liver dysfunction, are entirely unexpected. The approved methazolamide product label and prescribing information state that methazolamide therapy is contraindicated in cases of marked kidney or liver disease or dysfunction, and use of methazolamide in patients with cirrhosis may precipitate the development of hepatic encephalopathy (Methazolamide (methazolamide) Tablet. Prescribing information. 2006. TEVA PHARMACEUTICALS USA).

TABLE 1-1
Baseline (day 0) demographic data for methazolamide (MTZ) clinical;
trial participants. Met = metformin.

| | All | | | | | |
|---|---|---|---|---|---|---|
| | Placebo | AH MTZ | | | | |
| | (Alone + | (Alone + | Placebo | MTZ | Placebo + | |
| Parameter | Met) | Met) | Alone | Alone | Met | MTZ + Met |
| No. | 39 | 37 | 20 | 15 | 19 | 22 |
| Male (female) | 22 (17) | 28 (9) | 9 (11) | 10 (5) | 13 (6) | 18 (4) |
| Age (yr) | — | | | | | |
| Mean ± SD | 63 ± 9 | 63 ± 9 | 64 ± 8 | 63 ± 10 | 61 ± 10 | 63 ± 9 |
| Median (range) | 63 (35-76) | 65 (32-76) | 65 (51-76) | 65 (32-75) | 62 (35-76) | 64 (45-76) |
| Metformin (mg/day) | | | | | | |
| Mean ± SD | — | — | — | — | 1387 ± 642 | 1545 ± 999 |
| Median (range) | — | — | — | — | 1000 (500-3000) | 1250 (500-4500) |
| Body weight (kg) | | | | | | |
| Mean ± SD | 90 ± 16 | 93 ± 14 | 90.2 ± 17.6 | 93.0 ± 13.7 | 90.5 ± 14.9 | 92.3 ± 15.1 |
| Median (range) | 90 (57-130) | 93 (66-124) | 95.1 (57.2-123.0) | 95.3 (65.6-107.4) | 89.9 (69.0-130.0) | 89.6 (67.4-124.0) |
| HbA_{1c} (%) | | | | | | |
| Mean ± SD | 7.4 ± 0.6 | 7.1 ± 0.7 | 7.2 ± 0.6 | 7.1 ± 1.0 | 7.6 ± 0.5^{a} | 7.2 ± 0.4 |
| Median (range) | 7.35 (6.4^{b}-8.4)^{c} | 6.9 (6.2-10.1^{c}) | 7.15 (6.4^{b}-8.3) | 6.7 (6.2^{b}-10.1^{c}) | 7.7 (6.7-8.4) | 7.1 (6.6-8.0) |
| ALT (U/L) | | | | | | |
| Mean ± SD | 33.9 ± 16.1 | 31.5 ± 15.3^{d} | 33.6 ± 17.3 | 33.1 ± 16.9 | 34.2 ± 15.2 | 30.4 ± 14.5 |
| Median (range) | 32 (3-83) | 27.5 (16-83) | 28 (3-83) | 29 (16-83) | 34 (15-70) | 26 (18-77) |

^{a}n = 18.
^{b}HbA_{1c} = 6.5% at screening visit prior to randomization.
^{c}HbA_{1c} 8.4 % at screening visit prior to randomization.
^{d}n = 36.

TABLE 2
ALT and Changes in ALT (ΔALT) from baseline (Day 0) to Week 12 and Week 24

| | All | | | | | |
|---|---|---|---|---|---|---|
| | Placebo | All MTZ | | | | |
| | (Alone + | (Alone + | Placebo | MTZ | Placebo + | MTZ + |
| Parameter | Met) | Met) | Alone | Alone | Met | Met |
| ALT Day 0 (U/L) | | | | | | |
| n | 39 | 36 | 20 | 15 | 19 | 21 |
| Mean ± SD | 33.9 ± 16.1 | 31.5 ± 15.3 | 33.6 ± 17.3 | 33.1 ± 16.9 | 34.2 ± 15.2 | 30.4 ± 14.5 |
| Median (range) | 32 (3-83) | 27.5 (16-83) | 28 (3-83) | 29 (16-83) | 34 (15-70) | 26 (18-77) |
| ALT Week 12 (U/L) | | | | | | |
| n | 31 | 33 | 15 | 14 | 16 | 19 |
| Mean ± SD | 39.1 ± 31.6 | 20.9 ± 9.8 | 44.0 ± 41.0 | 22.1 ± 10.1 | 34.4 ± 19.7 | 19.4 ± 9.6 |
| Median (range) | 32 (15-187) | 19 (8-50) | 32 (23-187) | 20 (9-43 | 31.5 (15-92) | 18 (8-50) |
| ΔALT Week 12 | | | | | | |
| n | 31 | 32 | 15 | 14 | 16 | 18 |
| Mean ± SD | +3.9 ± 25.3 | −10.9 ± 7.7 | +7.7 ± 34.7 | −10.4 ± 9.3 | +0.4 ± 11.4 | −11.2 ± 6.4 |
| Median (range) | 0 (−17, +130) | −9 (−40, −2) | 0 (−17, +130) | −8.5 (−40, −4) | −0.5 (−13, +31) | −10 (−27, −2) |
| MTZ-Placebo | — | −14.8*^{§} | | -18.1 | | −11.6^{§} |
| ALT Week 24 (U/L) | | | | | | |
| n | 37 | 33 | 19 | 13 | 38 | 20 |
| Mean ± SD | 32.8 ± 13.2 | 21.3 ± 12.3 | 32.3 ± 11.6 | 22.0 ± 12.6 | 33.4 ± 15.0 | 20.8 ± 12.4 |
| Median (range) | 30 (15-63) | 20 (7-64) | 30 (15-51) | 17 (9-49) | 32 (16-63) | 20 (7-64) |
| ΔALT Week 24 | | | | | | |
| n | 37 | 32 | 19 | 13 | 18 | 19 |
| Mean ± SD | −1.4 ± 11.6 | −10.7 ± 8.2 | −3.0 ± 13.0 | −11.0 ± 8.1 | +0.2 ± 10.1 | −10.5 ± 8.5 |
| Median (range) | −1 (−32, +34) | −8.5 (−34, +4) | −3 (−32, +25) | −8 (−34, −3) | 0 (+16, +34) | −10 (−33, +4) |
| MTZ-Placebo | | −9.3^{†§} | | −8.0 | | −10.7^{§} |

MTZ = methazolamide;
Met = metformin;
ANCOVA = analysis of covariance
*Treatment effect MTZ-placebo (ANCOVA) = −15.9 (95% CI −25.4, −6.3) p = 0.0008
^{§}p < 0.005 vs. Placebo (ANOVA and unpaired, 2-sided t-test).
^{†}Treatment effect MTZ-placebo (ANCOVA) = −10.1 (95% CI −14.0, −6.1) p < 0.0001

### Example 2

### Effects of Methazolamide on Liver Lipid in Db/Db Mice

All reagents were purchased from Sigma-Aldrich (Australia). Dosing solutions of methazolamide were prepared fresh daily in sterile saline:PEG400 at 65:35 (v/v), protected from light and stored at room temperature. Male db/db mice (Animal Resource Centre, Australia) were housed with free access to water and food (standard rodent diet: Barastoc Rat & Mouse, Ridley Agriproducts, Australia). Room temperature was maintained at 21±2° C., humidity 40-70%, with a 12 h light/dark cycle. Mice were treated with methazolamide (50 mg/kg/day) or vehicle (n=4 per group) by single oral gavage doses each day for 9 days.

Daily blood samples were obtained from the tail tip of each mouse and glucose levels measured using a glucometer (AccuCheck II; Roche, Australia). At the end of the study, the animals were humanely killed and a portion of liver tissue (left lobe) was removed and fixed in 10% neutral-buffered formalin. The liver tissue was paraffin-embedded, sectioned (5 μm), mounted and stained with hematoxylin and eosin.

A separate portion of the liver (right lobe) was used to measure hepatic lipid content. Lipid was extracted using a modified Folch protocol. The tissue was homogenised in 2:1 chloroform/methanol solution (10 ml), and filtered into a 15 ml glass centrifuge tube. An additional 5 ml of 2:1 chloroform/methanol solution was added, followed by 2.5 ml of 0.9% NaCl. After thorough mixing, the extract was centrifuged for 5 min at 2,000 g at 10° C. After discarding the aqueous layer, the organic layer was dried under nitrogen, and total lipid content was assessed by weighing.

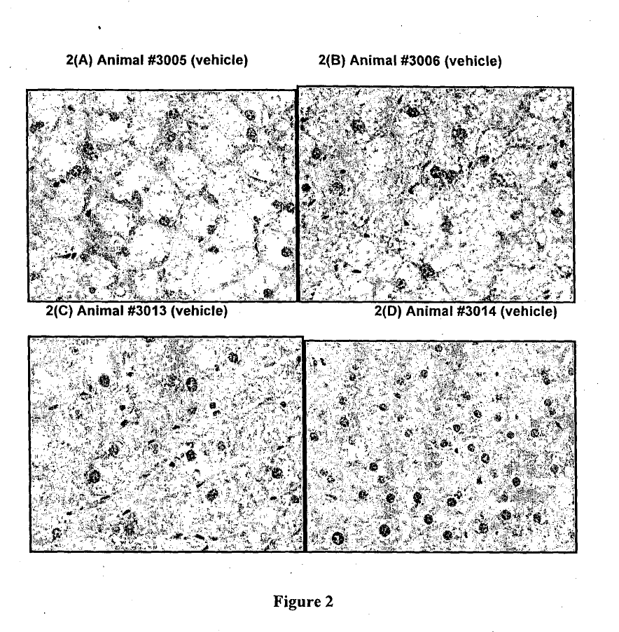

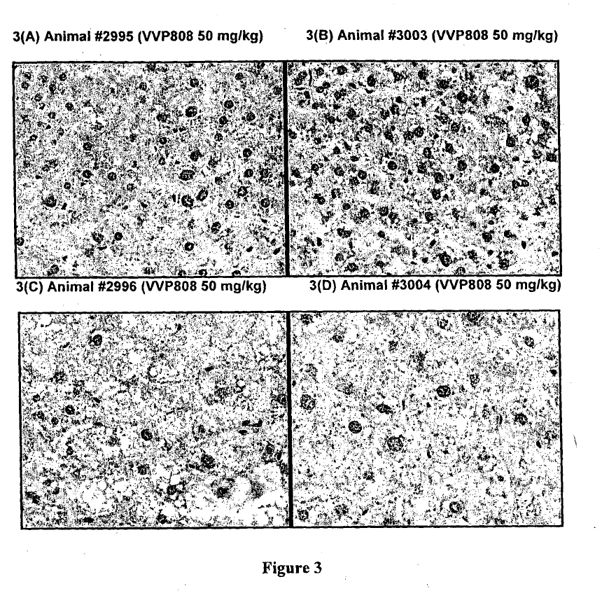

The results are depicted in Table 2-1 and in FIGS. 2 and 3.
1. Methazolamide treatment reduced fasting blood glucose levels by 47% relative to vehicle treated-controls.
2. Body weight tended to be lower (˜6%) in vehicle-treated animals, but this was not significant. The change in body weight over the 9 day dosing period was different between the groups; methazolamide-treated animals lost weight and vehicle-treated animals gained weight.
3. After 9 days of treatment, liver lipid content (w/w) was 43% lower in methazolamide-treated animals compared to vehicle treated controls.
4. Liver histology (FIG. 2) showed a difference between methazolamide- and vehicle-treated animals:
   3 of the 4 vehicle-treated animals had a high degree of hepatic steatosis.

Compared with images from the literature, these particular db/db mice appeared to have a relatively severe case of fatty liver disease.
2 of the 4 methazolamide treated db/db mice appeared to have greatly reduced hepatic steatosis.

TABLE 2-1

| | | |
|---|---|---|
| PARAMETER | Vehicle-treated | Methazolamide-treated |

Fasting Blood Glucose (mM)

| | | |
|---|---|---|
| Day 0 | 26.9 ± 1.6 | 26.0 ± 1.2 |
| Day 9 | 28.2 ± 1.6* | 13.7 ± 2.4* |

Body Weight (g)

| | | |
|---|---|---|
| Day 0 | 39.9 ± 1.7 | 41.6 ± 3.5 |
| Day 9 | 42.3 ± 1.9 | 39.0 ± 4.4 |

Change in Body Weight (g)

| | | |
|---|---|---|
| Day 9-Day 0 | +2.2 ± 0.7 | −2.6 ± 1.4^{§} |

Liver lipid content (% of liver weight)

| | | |
|---|---|---|
| Day 9 | 14.2 ± 3.2% | 7.4 ± 1.2%^{§} |

Groups were compared using a using a two-sided t-test.
*Statistically different from Day 0 (p < 0.05).
^{§}Statistically different from vehicle-treated animals (p < 0.05).

1. A method of decreasing serum ALT levels, in a patient in need thereof comprising administering an effective amount of methazolamide to said patient.

2. A method of treating or preventing liver dysfunction in a patient in need thereof, comprising administering an effective amount of methazolamide to said patient.

3. A method for reducing liver lipid content in patient in need thereof, comprising administering an effective amount of methazolamide to said patient.

4. A method for treating or preventing NAFLD in a patient in need thereof comprising administering an effective amount of methazolamide to said patient.

5. The method according to claim 4 for treating or preventing NAFL.

6. The method according to claim 4 for treating or preventing NASH.

7. The method according to claim 1 wherein the patient suffers from elevated ALT levels.

8. The method according to claim 1 wherein the patient is also pre-diabetic or diabetic.

9. The method according to claim 8 wherein the patient has an HbA_{1c} level of ≧6.5%.

10. The method according to claim 1 wherein the methazolamide is administered in combination with an anti-diabetic agent.

11. The method according to claim 10 wherein the anti-diabetic agent is metformin or a pharmaceutically acceptable salt thereof.

12. Use of methazolamide in the manufacture of a medicament for decreasing serum ALT levels in a patient.

13. Use of methazolamide in the manufacture of a medicament for treating or preventing liver dysfunction in a patient.

14. Use of methazolamide in the manufacture of a medicament for reducing liver lipid content in patient.

15. Use of methazolamide in the manufacture of a medicament for treating or preventing NAFLD.

16. The use according to claim 15 for treating or preventing NAFL.

17. The use according to claim 15 for treating or preventing NASH.

18. The use according to claim 1 wherein the patient is also pre-diabetic or diabetic.

19. The use according to claim 1 wherein the patient has an HbA_{1c} level of ≧6.5%.

20. The use according to claim 1 wherein the methazolamide is administered in combination with an anti-diabetic agent.

21. The use according to claim 20 wherein the anti-diabetic agent is metformin or a pharmaceutically acceptable salt thereof.

22. A composition for treating or preventing liver dysfunction and/or lowering ALT levels and/or reducing liver lipid levels in a patient said composition comprising methazolamide, together with one or more pharmaceutically acceptable additives.

23. A combination for use in treating or preventing liver dysfunction and/or lowering ALT levels and/or reducing liver lipid levels in a patient undergoing treatment with an anti-diabetic agent, said combination comprising methazolamide and an anti-diabetic agent.
